(19) United States
(12) Patent Application Publication
(10) Pub. No.: US 2013/0090713 A1
(43) Pub. Date: Apr. 11, 2013

(54) RADIALLY EXPANDABLE STENT

(71) Applicant: Thomas Nissl, Adendorf (DE)

(72) Inventor: Thomas Nissl, Adendorf (DE)

(21) Appl. No.: 13/581,963

(22) PCT Filed: Feb. 28, 2011
(86) PCT No.: PCT/EP2011/000958

§ 371 Date: Dec. 13, 2012

(30) Foreign Application Priority Data
Mar. 1, 2010 (DE) 10 2010 009 802.7

(51) Int. Cl. A61F 2/90

(52) U.S. Cl. CPC A61F 2/90
USPC 623/1.2

(57) ABSTRACT

The invention relates to a radially expandable vessel support (100) comprising a supporting body (102) having a plurality of longitudinally arranged ring segments as well as terminal supporting segments (103) and at least one terminal edge segment (104) arranged at a terminal supporting segment (103), with the terminal supporting segments (103) having a meandering configuration with a multitude of supporting arches (108) and the edge segment or segments (104) having a plurality of relief arches (106), with the number of the relief arches (106) being lower than the number of the supporting arches (108) and the relief arches (106) beginning at the peripheral/edge-situated reversal points (116) of the supporting arches (108).

The invention relates to a radially expandable stent, particularly for use inside an organ performing frequent spontaneous movements.

Stents, also termed vessel or vascular supports, are medical implants inserted into hollow organs with a view to keeping them open. For this purpose, tubular lattice structures are normally used which are introduced into the relevant hollow organs in compressed form, the diameter of which is then radially expanded at the placement site to such an extent that it matches the inner walls of the relevant organs.

An example of such a stent has been disclosed in publication DE 10 2007 030 753 A1 describing a stent having a tubular lattice structure. Another stent is known, for example, from DE 699 20 457 T2, and common to all these stents is that they are designed to show a relevant lattice structure formed by a multitude of meandering or zigzagging ring elements arranged one after the other in relation to the longitudinal axis.

This, however, means that the end regions of stents of this nature are formed by segments terminating in more or less pointed elements which are pressed against the inside of the respective hollow organs in a rigid manner. In case these hollow organs show high autonomous mobility such as for example the intestinal tract (duodenum, colon), the air tube (trachea), the gullet (esophagus) or relevant usually non-coronary blood vessels any movement of these organs will cause high mechanical loads to be exerted on the respective organ walls in the longitudinal edge area of the vessel support due to the pointed end segments. This may not only lead to an unpleasant irritation of the organs but also severely injure the respective organ. Moreover, any possible option to displace the vessel support subsequently inside the organ will be rather limited because of the danger that always exists due to the pointedly terminating segments getting caught or hooked up at the organ walls or even penetrating into the organ walls during the relocation (displacement process) of the vessel support. A contributory factor here is the high radial force exerted by the edge or terminal segments.

Consequently, the objective of the present invention is to provide an improved stent the terminal areas of which are atraumatically designed to exert reduced radial forces.

To achieve this objective the invention relates to a radially expandable stent comprising a supporting body having a plurality of longitudinally arranged ring segments as well as terminal supporting segments and at least one terminal edge segment arranged at a terminal supporting segment, with the terminal supporting segments having a meandering configuration with a multitude of supporting arches and the edge segment or segments having a plurality of relief arches, with the number of the relief arches being lower than the number of the supporting arches and the relief arches beginning at the peripheral/edge-situated reversal points of the supporting arches.

The terms “stent” and “vessel support” are used synonymously hereinafter.

The inventive vessel support is provided with a customary supporting body consisting of a plurality of ring segments arranged next to each other in a row in longitudinal direction. Attached to these central ring segments are terminal supporting segments having a meandering configuration and comprising a plurality of supporting arches. Structure and design of the ring segments arranged between these terminal supporting segments are of conventional and optional nature. Preferably, meandering ring segments are to be used here as well, i.e. ring segments designed to show a meandering, zigzagging or serpentine line configuration.

Attached to at least one of the two terminal supporting segments is a peripheral segment comprising a plurality of relief arches. These relief arches are designed so as to create an atraumatic and flexible end region of the vessel support and in this way avoid tissue irritations and injuries.

According to the invention, the number of the relief arches is lower than the number of the supporting arches, with the relief arches starting out at the peripheral reversal points of the supporting arches. Preferably, the starting/end points of adjacent relief arches are located on neighboring reversal points.

Reducing the number of relief arches provided will for one thing enhance the flexibility of the edge segment or segments and also bring down the radial forces acting on the hollow organ when expanded or stabilized by the stent. Since the number of supporting arches of the terminal supporting segments is greater they exert of course higher radial forces. Therefore, it is to be understood that between its connecting points each relief arch spans several supporting arches.

Expediently, the number of supporting arches is an integer multiple of the number of relief arches. In this context, a relief arch may span two three, four or even five supporting arches so that the flexibility and radial force of the edge segments can be finely adjusted.

The relief arches are usually attached to the peripheral reversal points of neighboring supporting arches. The relief arches may be designed in such a way that they do not intersect.

However, preferred is an arrangement of relief arches that provides for neighboring relief arches to be joined at points of intersection. In particular, these points of intersection are mechanically connected with each other. The connection points may be designed in the form of eyelets.

Radial force and flexibility of the relief arches can be influenced in the way in which the intersections are arranged. The more distant the intersections are located from the supporting body of the vessel support the stiffer the edge segment becomes. It is usually considered expedient to arrange the intersections in the half of the edge segment adjoining the supporting body which means the points, of intersection are situated nearer to the supporting body than to the end of the edge segments. The starting and end points of the relief arches of the edge segments are expediently located at the reversal points of the supporting arches. Such starting and end points are preferably arranged on supporting arches that are provided with connecting elements forming a junction with the central segments of the supporting body, i.e. that connect the ring segments with each other. These connecting elements are structural webs of the vessel support intended to both counteract a length contraction when the vessel support is expanded and firmly connect the ring segments with each other. The connecting elements, in particular, start at the inside of the reversal points of the supporting arches from where they extend—with the vessel support in non-expanded state—parallelly to the webs of the supporting segment or ring segments towards the adjacent ring segment.

As per a special embodiment of the invention additional eyelets may be arranged in the relief arches at their starting and end points located on the reversal points of the supporting arches.

The eyelets arranged at the points of intersections as well as starting/end points of the relief arches may, for example, accommodate marker elements consisting of radiopaque material and facilitating placement of the vessel support. Suitable marker elements may, in particular, be gold and platinum metals as well as alloys thereof but also other materials having radiopaque properties.

Furthermore, the eyelets may serve to secure the vessel support in the event the support is crimped onto a balloon with a view to expand it within the hollow vessel hydraulically. It is also possible to arrange for a filament, thread or wire to be run through the eyelets, e.g. for the purpose of again contracting/retracting the vessel support during placement which may in particular be expedient to allow for corrections in case of inaccurate placements or broken webs to enable the vessel support to be drawn back into a placement catheter.

The flexibility and radial force of the edge segments may also be influenced by appropriately selecting the width of the webs, i.e. a reduced web width results in lower radial forces and higher flexibility. It is also possible to provide individual arches of stronger design in terms of web width with a view to creating in this manner an attachment point for a retriever by means of which the vessel support can be explanted in the event it has to be replaced or is no longer required. Usually, the relief arches are provided with webs of a width smaller than that of the segments of the supporting body.

The inventive stents respectively vessel supports are manufactured in a customary manner, for example by means of laser cutting out of an appropriately dimensioned tube. Basically however, vessel supports made of wire may also be employed. Materials appropriate for the purpose are in particular shape-memory materials such as nitinol but also medically usable customary steel grades, particularly spring steel material.

Especially when intended for use as vascular support the vessel support according to the invention is to be crimped onto a customary balloon so that it can be transferred by means of a catheter to the placement site where it is hydraulically expanded until the required diameter has been reached. When the support has been expanded after implantation/placement has taken place correctly the balloon catheter is retracted, with any threads that may exist in the eyelets being drawn back and removed as well. In the event a replacement is necessary the stent edge can be contracted by means of the thread run through the eyelets arranged at the points of intersection or starting/end points and the stent withdrawn into the catheter and then placed in position again.

It goes without saying that placement of an inventive vessel support made of a self-expanding material, for example the shape-memory alloy nitinol, can be performed without having to use a balloon.

Moreover, the invention also relates to stents and vessel supports crimped onto balloons as well as balloon catheters provided with inventive stents and vessel supports.

Vessel supports and stents according to the invention may be implanted into a variety of body-own hollow organs. They may be used in particular in the region of the duodenum, the esophagus, the trachea, ureters, bile ducts but also in blood vessels especially in the peripheral area.

It is to be understood that as considered necessary for a given case the inventive vessel supports or stents may be provided on one or both ends with edge segments having relief arches. As a rule, it will be expedient to provide both ends with these atraumatic edge segments.

Stents according to the invention offer advantages in that the arches absorb in a planar fashion forces that are caused by the walls of the organs into which the vessel support has been implanted and act on the end of the vessel support. The arches thus serve to cushion forces arising in the edge area of and acting on the vessel support as a result of the movements the organs perform. Moreover, due to the arch-shaped configuration of the edge segments an irritation of the organ or penetration of the ends of the vessel support into the walls of the organ is avoided.

On account of the reduced radial forces and stiffness of the edge segments in comparison to the radial forces and stiffness of the supporting body exclusively the supporting body serves to stabilize or shore up the relevant hollow organs. The edge segments are thus merely intended to absorb and transmit forces caused by the organ and acting on the vessel support in its peripheral region, primarily in longitudinal direction of the support. This makes it possible, for example, for the edge segments to follow the movements of the organ into which the vessel support has been inserted and thus create a soft transmission of forces between the organ and the supporting body.

The manner in which the edge segments are connecting to the terminal supporting segments and the arrangement of points of intersection in neighboring relief arches offers benefits in that any forces acting on the arches are transmitted directly to locations of the supporting body where the maximum mechanical stability of the supporting body exists. Even in case of severe deformation or great movements of the walls of the organ into which the vessel support has been implanted no irreversible and, more important, undesired deformation of the supporting body occurs since, for example, forces absorbed by the relief segments are prevented from having a deformative effect on the supporting body.

Preferred embodiments of the invention are illustrated hereinafter by way of the following drawings where

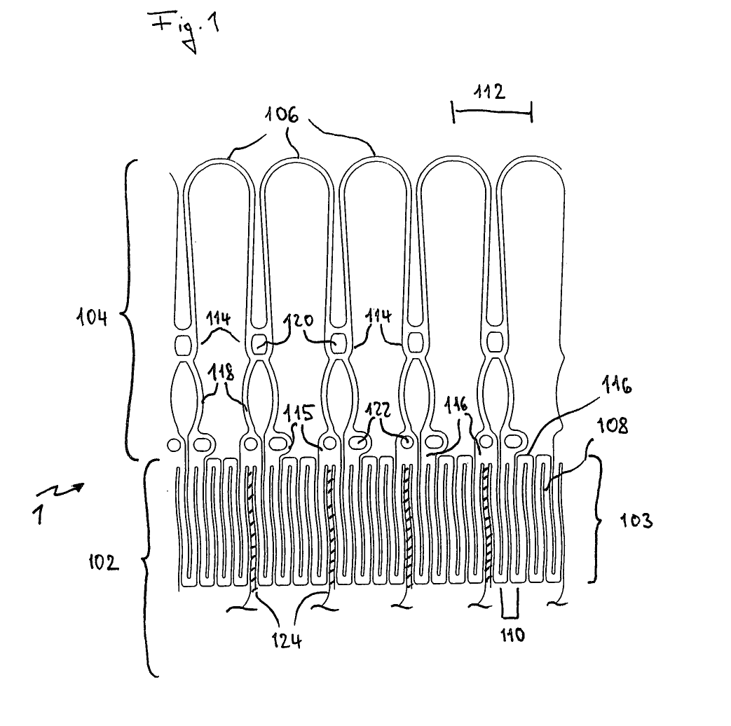

FIG. 1: shows a developed representation of an embodiment of a vessel support,

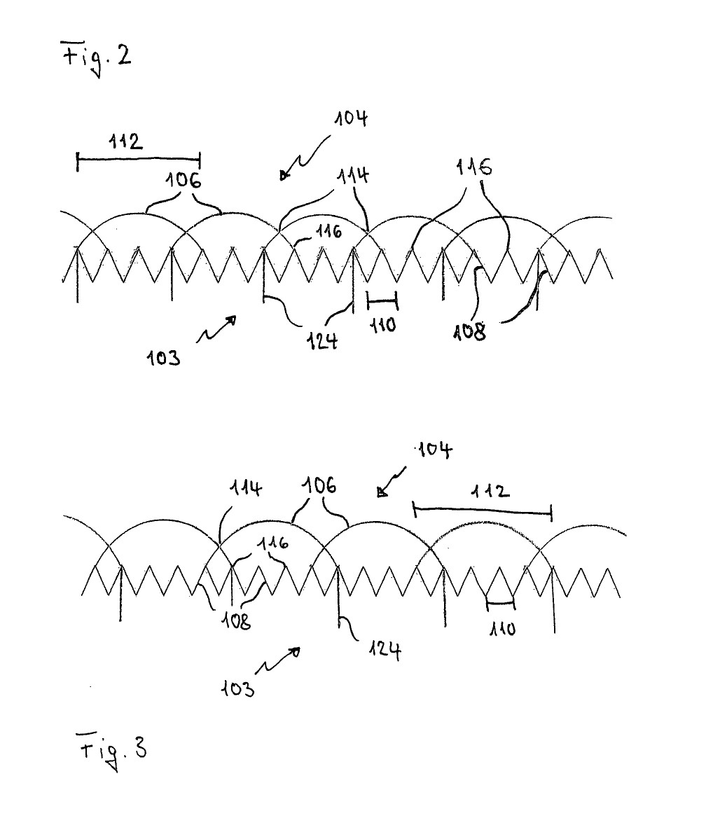

FIG. 2: is a schematic view of a relief segment arranged on a terminal supporting body, and

FIG. 3: is another schematic view of a relief segment arranged on a supporting body.

In the text hereunder similar or alike elements are identified by the same reference signs.

FIG. 1 shows a schematic view of the end region of stent 100 according to the invention in developed, non-expanded state. Vessel support 100 is a radially expandable stent comprising an elongated supporting body 102, with only one terminal supporting segment 103 of this elongated supporting body 102 being shown, such terminal segment 103 being provided with a plurality of meandering supporting arches 108 resulting in the supporting segment to assume an undulated form in circumferential direction. Moreover, vessel support 100 is provided with a ring-shaped edge segment 104, with such edge segment 104 being arranged on one end of the elongated supporting body 102 and comprising a plurality of curved arches 106 arranged in the circumferential direction of the ring-shaped relief body. It is to be understood here that another edge segment may be arranged on the other end of the supporting body 102 in the same manner.

In the embodiment shown in FIG. 1 the web width of the arches (106) is slightly reduced in comparison to the material thickness of the supporting segment 103. However, this configuration is not obligatory and can be varied to achieve the desired stiffness of the supporting body and relief body.

As can also be seen from FIG. 1, elements 103 of the ring segment are curved in circumferential direction at a first period length 110 and the arches 106 of the relief body 104 at a second period length of 112. Since period length 112 is greater than period length 110 the stiffness of the edge segment 104 is lower than that of the supporting body 102. In consequence, the number of supporting arches is higher by a factor of 5 than the number of the relief arches 106.

The undulated supporting segment 103 directly arranged on the edge segment 104 comprises a plurality of reversal points facing the edge segment 104. Moreover, the arches 106 have a plurality of starting and end points 115 which are facing the supporting body 102. The arrangement of the edge segment 104 on the supporting segment 103 is brought about by the starting and end points 115 having their origin at the reversal points 116.

The arches 106 of the edge segment 104 cross each other at points of intersection 114. Points of intersection 114—here provided with eyelets 120 which will be dealt with more closely later—serve to distribute the forces exerted on arches 106 onto the stent-side webs 118 the starting and end points 115 of which terminating at the reversal points 116 of the supporting segment 103. The intersection locations 114 are arranged nearer to the supporting segment 103 than to the outer edge of the edge segment 104. By displacing the position of points of intersection 114 in relation to the supporting segment 103 the flexibility and stiffness of the edge segment can be varied as desired. It is generally true that the distance between the points of intersection 114 and the end of the stent 100 is greater than from the outer edge of the supporting segment 103.

As shown in the embodiment of FIG. 1 the overall stiffness increases gradually from the outer end of the edge segment 104 to the supporting segment 103. Arches 106 absorb forces in a mechanically “soft” manner and may be reversibly deformed during this process. At the points of intersection 114 these forces are distributed over the webs 118 whose stiffness is higher than that of arches 106.

Finally, the forces exerted via the webs 118 are transmitted at reversal points 116 on to the undulated elements of the supporting segment 103.

As mentioned above, FIG. 1 only shows a single ring segment 103 of the supporting body 102. In actual practice the supporting body 102 will comprise several ring segments arranged in succession, with such ring segments being mechanically joined by means of suitable longitudinal connecting elements 124. In the embodiment shown in FIG. 1 the longitudinal connecting elements 124 are arranged at the reversal points 116 of arches 108 where the arches 106 commence, respectively end. In this manner, the forces acting on the arches 106 of the edge segment 104 are not only transmitted to the single supporting segment 103 as shown in FIG. 1 but also to ring segments adjacent to this said ring segment of the supporting body so that a uniform distribution of forces is achieved throughout vessel support 100.

Also illustrated in FIG. 1 are eyelets 120 arranged at points of intersection 114 as well as eyelets 122 arranged at the starting/end points 116. These eyelets 120 and 122 may in general be used to accommodate imaging markers and/or repositioning elements such as for example a wire or thread. In particular, these components can be employed to enable the vessel support 100 to be easily displaced inside an organ due to the fact that the diameter of the edge segment can be reduced even after the support has been placed in position and in this manner facilitate shifting the stent or retracting it into a catheter.

FIG. 2 is a schematic view of a vessel support 100 with an edge segment 104 and a supporting segment 103. Supporting body 102 comprises, inter alia, a ring segment of zigzagging configuration. The undulated arches 106 of the edge segment 104 are in this case also located at the reversal points 116 of the supporting body 103. In the embodiment shown in FIG. 2 the reversal points 116 are formed by the pointed ends of the ring segment facing the edge segment 104. Important to note in FIG. 2 is that the period length 110 which determines the degree of curvature of the ring segments in circumferential direction is significantly shorter than period length 112 determining the curvature of the arches of the relief body 104. Supporting arches 108 are arranged at a ratio of 3:1 with respect to the relief arches 108. Arches 106 are connected with each other at points of intersection 114.

In this way a desired mechanical stiffness of the edge segment 104 can be achieved which is lower than the stiffness of supporting segment 103. Support of the organ walls is exclusively brought about as a result of the stiffness of supporting body 102, with the minor stiffness of the edge segment 104 exclusively serving to absorb deformation forces arising from movements of the organ and in this manner accomplish shielding the pointed segments (reversal points 108) of supporting body 102 in longitudinal direction of vessel support 100.

In comparison with FIG. 2 the stiffness of the relief body 104 was further reduced in FIG. 3 in reference to the stiffness of supporting body 102. This was achieved through further shortening period length 112 and further increasing period length 112. In this manner the ratio between period length 112 and period length 110 was increased resulting in the stiffness of edge segment 104 reducing in respect of the stiffness of supporting segment 102. The ratio of the number of supporting arches 108 to the number of relief arches is 4:1.

Accordingly, as shown in FIG. 2 one arch 106 always straddles three reversal points 108 whereas in FIG. 3 always four reversal points 108 are spanned by one arch 106.

### LIST OF REFERENCE SIGNS

100 Vessel support
102 Supporting body
103 Terminal supporting segment
104 Edge segment
106 Relief arch
108 Supporting arch
110 Period in supporting segment
112 Period in edge segment
114 Point of intersection
115 Starting/end point of arches 106
116 Reversal point
118 Web
120 Eyelet
122 Eyelet
124 Longitudinal connecting element

1. Radially expandable stent (100) comprising a supporting body (102) having a plurality of longitudinally arranged ring segments as well as terminal supporting segments (103) and at least one terminal edge segment (104) arranged at a terminal supporting segment (103), with the terminal supporting segments (103) having a meandering configuration with a multitude of supporting arches (108) and the edge segment or segments (104) having a plurality of relief arches (106),
characterized in that the number of the relief arches(106) is lower than the number of the supporting arches (108) and the relief arches (106) begin at the peripheral/edge-situated reversal points (116) of the supporting arches (108).

2. Stent according to claim 1, characterized in that the number of the supporting arches (108) is an integer multiple of the number of relief arches (106).

3. Stent according to claim 2, characterized in that the integer multiple is a number ranging between 2 and 5.

4. Stent according to claim 1, characterized in that neighboring relief arches (106) cross each other at points of intersection (114).

5. Stent according to claim 4, characterized in that relief arches (106) are connected at the points of intersection (114).

6. Stent according to claim 4, characterized in that the points of intersection (114) are formed in the shape of eyelets (120).

7. Stent according to claim 4, characterized in that the distance between points of intersection (114) and supporting segment (103) is lower than to the end of the edge segment (104).

8. Stent according to claim 1, characterized in that the relief arches (106) start and/or end at reversal points (116) of the supporting arches (108) which are provided with connecting elements (124) forming a junction with the central segments of the supporting body (102).

9. Stent according to claim 8, characterized in that the connecting elements (124) start at the inside of the reversal points (116) of the supporting arches (108).

10. Stent according to claim 1, characterized in that the relief arches (106) are provided with eyelets (122) in the area of the start/end locations of the relief arches (106) at the reversal points (116) of the supporting arches (108).

11. Stent according to claim 6, characterized by marker elements in the eyelets (120, 122) of points of intersection (114) or start/end locations of relief arches (106) at the reversal points (116).

12. Stent according to claim 1, characterized in that the web width of the relief arches (106) is smaller than the web width of the supporting arches.

13. Stent according to claim 1, characterized in that the web width of individual relief arches (106) is greater than that of the other relief arches (106).

14. Stent according to claim 1, characterized in that it is provided with a thread extending through the eyelets (120 or 122).

15. Stent according to claim 1 crimped onto a balloon.
